(19) United States
(12) Patent Application Publication
(10) Pub. No.: US 2024/0169218 A1
(43) Pub. Date: May 23, 2024

(54) SYSTEM AND METHOD FOR CONCISE ASSESSMENT GENERATION USING MACHINE LEARNING

(71) Applicant: Kang LEE, Toronto (CA)

(72) Inventor: Kang LEE, Toronto (CA)

(21) Appl. No.: 18/552,812

(22) PCT Filed: Mar. 29, 2022
(86) PCT No.: PCT/CA2022/050469

§ 371 Date: Sep. 27, 2023

Related U.S. Application Data
(60) Provisional application No. 63/169,671, filed on Apr. 1, 2021.

(51) Int. Cl. G06N 5/022

(52) U.S. Cl. CPC G06N 5/022

(57) ABSTRACT

A system and method for concise assessment generation using machine learning. The method including: receiving an input dataset including completed sample assessments, the sample assessments each include assessment features for determining an output status and an associated likelihood of the output status; selecting a subset of the assessment features by determining which assessment features of the sample assessment weigh more heavily in predicting the output status; predicting, using a machine learning model, a quantity of assessment features required to achieve at least a predetermined classification accuracy of an output of the assessment, the machine learning model trained using the selected subset of assessment features and the weighting of such assessment features; and outputting one or more concise assessments, each concise assessment including the quantity of assessment features and having a classification accuracy of at least the predetermined classification accuracy.

### TECHNICAL FIELD

The present disclosure relates generally to optimization using machine learning. More particularly, the present disclosure relates to a system and method for concise assessment generation using machine learning.

### BACKGROUND

There are many forms of assessments to measure people's abilities, attitudes, opinions, skills, and wellbeing. In many situations, the assessments consist of many items and will take over 10 or 20 minutes, or even hours to complete. Such assessments in general can provide reliable and valid measurements of what the assessments are designed to assess if the participants respond to all items. However, frequently participants may not have time or patience to complete all items for various reasons, resulting in either fewer participants being willing to complete the assessment or poor quality of the assessment if the participants are forced to complete the whole assessment.

### SUMMARY

In an aspect, there is provided a method for concise assessment generation using machine learning, the method executed on one or more processors, the method comprising: receiving an input dataset comprising completed sample assessments, the sample assessments each comprise assessment features for determining an output status and an associated likelihood of the output status; selecting a subset of the assessment features by determining which assessment features of the sample assessment weigh more heavily in predicting the output status; predicting, using a machine learning model, a quantity of assessment features required to achieve at least a predetermined classification accuracy of an output of the assessment, the machine learning model trained using the selected subset of assessment features and the weighting of such assessment features; and outputting one or more concise assessments, each concise assessment comprising the quantity of assessment features and having a classification accuracy of at least the predetermined classification accuracy.

In a particular case of the method, the method further comprising encoding and normalizing the assessment features.

In another case of the method, the method further comprising up-sampling positive sample assessments to match the number of negative sample assessments.

In yet another case of the method, selecting a subset of the assessment features comprises performing Minimum Redundancy Maximum Relevance (MRMR) feature selection using Mutual Information Quotient (MIQ) criteria.

In yet another case of the method, selecting a subset of the assessment features further comprises performing feature selection by fitting an Extra Tree Classifier for the assessment features and ranking importance of the assessment features.

In yet another case of the method, the MRMR feature selection is combined with the Extra Tree Classifier feature selection.

In yet another case of the method, the machine learning model is trained using multiple different permutations of assessment features from the selected subset.

In yet another case of the method, training of the machine learning model begins with permutations of one of the assessment features followed by permutations of increasing numbers of assessment features until the predetermined accuracy is achieved.

In yet another case of the method, the one or more concise assessments comprise a compendium of all the concise assessments that comprise the quantity of assessment features and have a classification accuracy of at least the predetermined classification accuracy

In yet another case of the method, classification accuracy of the concise assessments is determined based on whether the classifications outputted by the concise assessments match, or substantially match within a predetermined tolerance, that obtained when all items of the sample assessment are used.

In another aspect, there is provided a system for concise assessment generation using machine learning, the system comprising one or more processors and a data storage, the data storage comprising instructions for the one or more processors to execute: a preprocessing module to receive an input dataset comprising completed sample assessments, the sample assessments each comprise assessment features for determining an output status and an associated likelihood of the output status; a selection module to select a subset of the assessment features by determining which assessment features of the sample assessment weigh more heavily in predicting the output status; a machine learning module to predict, using a machine learning model, a quantity of assessment features required to achieve at least a predetermined classification accuracy of an output of the assessment, the machine learning model trained using the selected subset of assessment features and the weighting of such assessment features; and an assessment module to output one or more concise assessments, each concise assessment comprising the quantity of assessment features and having a classification accuracy of at least the predetermined classification accuracy.

In a particular case of the system, the selection module further encodes and normalizes the assessment features.

In another case of the system, the selection module further up-samples positive sample assessments to match the number of negative sample assessments.

In yet another case of the system, selecting a subset of the assessment features comprises performing Minimum Redundancy Maximum Relevance (MRMR) feature selection using Mutual Information Quotient (MIQ) criteria.

In yet another case of the system, selecting a subset of the assessment features further comprises performing feature selection by fitting an Extra Tree Classifier for the assessment features and ranking importance of the assessment features.

In yet another case of the system, the MRMR feature selection is combined with the Extra Tree Classifier feature selection.

In yet another case of the system, the machine learning model is trained using multiple different permutations of assessment features from the selected subset.

In yet another case of the system, training of the machine learning model begins with permutations of one of the assessment features followed by permutations of increasing numbers of assessment features until the predetermined classification accuracy is achieved.

In yet another case of the system, the one or more concise assessments comprise a compendium of all the concise assessments that comprise the quantity of assessment features and have a classification accuracy of at least the predetermined classification accuracy

In yet another case of the system, classification accuracy of the concise assessments is determined based on whether the classifications outputted by the concise assessments match, or substantially match within a predetermined tolerance, that obtained when all items of the sample assessment are used.

These and other aspects are contemplated and described herein. It will be appreciated that the foregoing summary sets out representative aspects of systems and methods to assist skilled readers in understanding the following detailed description.

### BRIEF DESCRIPTION OF THE DRAWINGS

The features of the invention will become more apparent in the following detailed description in which reference is made to the appended drawings wherein:

FIG. 1 is a block diagram of a system for concise assessment generation using machine learning, in accordance with an embodiment;

FIG. 2 is a flowchart of a method for concise assessment generation using machine learning, in accordance with an embodiment;

FIG. 3 is a chart illustrating Area Under Curve (AUC) scores of Receiver Operating Characteristic (ROC) curve for various machine learning models, averaged over 10 permutations of different numbers of questions, according to an example experiment in accordance with the system of FIG. 1; and

FIG. 4 is a chart illustrating test F1 scores for all models, averaged over 10 permutations of different numbers of questions, according to an example experiment in accordance with the system of FIG. 1.

### DETAILED DESCRIPTION

Embodiments will now be described with reference to the figures. For simplicity and clarity of illustration, where considered appropriate, reference numerals may be repeated among the Figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

Any module, unit, component, server, computer, terminal, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors.

The present embodiments advantageously provide an approach to assess participants using machine learning to generate the assessment. The present embodiments take advantage of several facts determined by the present inventors that: (1) many items in almost all assessments are redundant in that multiple items correlate highly with each other and thus obtain similar information from the participants; and (2) machine learning is able to obtain non-linear combinations of participants' responses to different items to maximize information needed from the participants. By capitalizing on these facts, the number of items needed to assess participants to achieve a similar level of accuracy can be reduced to that when all items of an assessment are used.

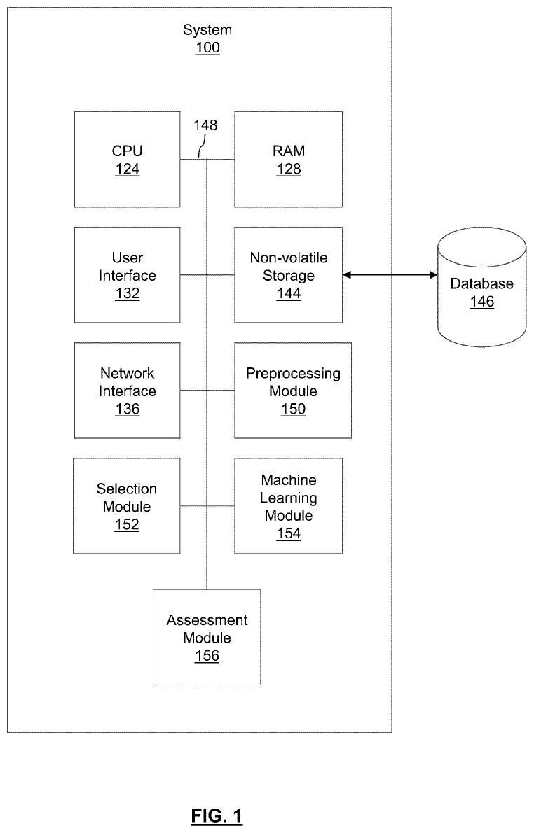

Turning to FIG. 1, shown therein is a diagram of a system for concise assessment generation using machine learning 100, in accordance with an embodiment. The system 100 can include a number of physical and logical components, including a central processing unit (“CPU”) 124, random access memory (“RAM”) storage 128, a user interface 132, a network interface 136, memory comprising non-volatile storage 144, and a local bus 148 enabling CPU 124 to communicate with the other components. CPU 124 can include one or more processors. RAM 128 provides relatively responsive volatile storage to CPU 124. The user interface 132 enables a user to provide input via, for example, a touchscreen. The user interface 132 can also output information to output devices, for example, to the touchscreen. Non-volatile storage 144 can store computer-executable instructions for implementing the system 100, as well as any derivative or other data. In some cases, this data can be stored or synced with a database 146, that can be local to the system 100 or remotely located (for example, a centralized server or cloud repository). During operation of the system 100, data may be retrieved from the non-volatile storage 144 and placed in RAM 128 to facilitate execution. In an embodiment, the CPU can be configured to execute various modules, for example, a preprocessing module 150, a selection module 152, a machine learning module 154, and an assessment module 156. In further cases, functions of the above modules can be combined or executed on other modules. In some cases, functions of the above modules can be executed on remote computing devices, such as centralized servers and cloud computing resources communicating over a network via the network interface 136.

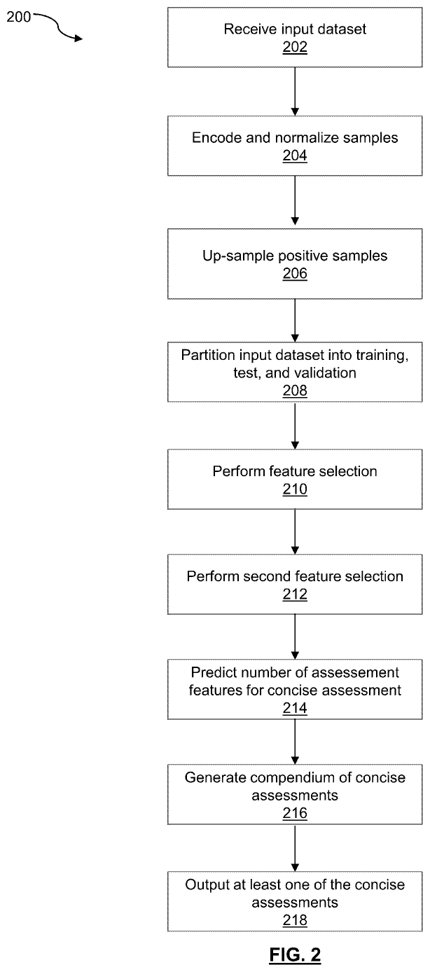

FIG. 2 is a flow chart for a method for concise assessment generation using machine learning 200, according to an embodiment. At block 202, the preprocessing module 150 receives an input dataset comprising a training collection of sample assessments for separate participants. The sample assessments comprise a quantity of assessment features for determining an output status. The assessment features can include, for example, questions, queries, demographic information, biographic information, and/or other elements.

At block 204, in some cases, the preprocessing module 150 encodes and normalizes features from the sample assessments; for example, using one-hot encoding and z-scores. At block 206, in some cases, the preprocessing module 150 up-samples positive assessment samples to match up with a number of negative samples. The up-sampling can randomly select rows from the specified categories and duplicates them in the dataset, so the row count matches the other categories. At block 208, in some cases, the preprocessing module 150 partitions the input dataset into training, test, and validation datasets.

At block 210, the selection module 152 performs feature selection by determining which features of the sample assessment weigh more heavily in predicting a particular output status. In most cases, the selection module 152 performs feature selection on the full input dataset. In an example, a Minimum Redundancy Maximum Relevance (MRMR) feature selection technique, using the Mutual Information Quotient (MIQ) criteria, can be applied to identify a subset of features that are most important for predicting the output status. MRMR is an unsupervised technique that selects the most relevant features based on pairwise correlations, or mutual information of each pair of variables in the dataset, while minimizing the redundancy between variables.

At block 212, to serve as a reference, the selection module 152 can also use a second feature selection approach by fitting, for example, an Extra Tree Classifier for all the features before ranking the most important features. This can be used to ensure the robustness of the feature selection by MRMR, and to validate the results. The Extra Tree Classifier fits several randomized decision trees (i.e., extra-trees) on sub-samples of the dataset and averages the results. The feature importance can be obtained, for example, using a normalized total reduction of the criterion brought by that feature, known as the Gini importance. Gini importance, also known as Mean Decrease in Impurity (MDI), determines each feature importance as the sum over the number of splits across all decision trees that include the feature, proportionally to the number of samples it splits. The features selected to be part of the subset can be obtained by ranking the feature importance. These results can then be combined with the subset from the MRMR selection to derive a subset for the concise assessment with combined feature importance rankings. The combination can use any suitable approach; for example, mean, median, addition, or the like. In some cases, the top n most important features (e.g., 10) can be used for training the machine learning model in block 214. Although more features can be used, limiting to the top n number of important features is advantageous to avoid combinatory explosion and to save the overall computational time.

At block 214, the machine learning module 154 predicts a number of assessment features required to achieve a predetermined accuracy of the output status, based on the output subset identified by the selection module 152, using one or more trained machine learning models. The one or more trained machine learning models take, as input features, the n most important features identified from the feature selection. Advantageously, the machine learning module 154 can be used to find a minimum number of questions that would be required to compose a sufficiently accurate assessment for predicting the output status. The machine learning module 154 trains the prediction by evaluating how many assessment features from the sample assessment it would take for the machine learning model to predict the presence of the output status at a predetermined accuracy level. The machine learning models can be trained for different permutations of assessment features selected from the output subset; in some cases, along with the demographic, biographic, or other elements.

Training of the machine learning model can start with permutations of one assessment feature, then permutations of increasing numbers of assessment features can be performed. In an example, a single feature can be randomly selected from the n most important features to train the machine learning models. The machine learning module 154 can evaluate the performance of the trained model to determine whether using the single feature for training provides a sufficiently accurate assessment. Where the model does not provide a sufficiently accurate assessment, successively more combinations of features can be used for training. Such as training models with combinations of two features by randomly selecting two features from the n most important features. The number of features used for training can be increased successively until the total number of n most important features is reached.

For training of the model, the input dataset can be split into training (e.g., 80%), test (e.g., 10%), and pristine validation datasets (e.g., 10%). The machine learning training is done on the training dataset only, and internally validated on the test dataset. After obtaining the model, the machine learning module 154 can recombine the training and testing datasets and partition them in the same way (80% vs. 10%) and train and test another model. This process of performing data recombination, partitioning, and training/testing can be repeated a number of times (e.g., at least 50 times) in order to produce a stable Gaussian distribution of model performance metrics. The models can then be tested using the validation dataset to assess how well these models performed. The holdout validation dataset is untouched during training of the models to ensure a fair comparison between models.

In some cases, the demographic, biographic, or other elements can be also used in the training dataset to the machine learning module 154. In some cases, the output label is a binary column representing the output status (0 or 1). In some other cases, the output label is multi-class where the output status represents a number of outcomes (e.g., 0, 1, 2, 3 . . . ). Various suitable machine learning models can be used. For example, Regression (linear or logistic), Support Vector Machine (SVM), Random Forest (RF), and Naïve Bayes. Further, Artificial Neural Networks (ANN) can be used, for example, Multilayer Perceptron (MLP) and Convolutional Neutral Network (CNN). Further, gradient boosting or ensemble techniques, such as XGBoost, can be used.

In some cases, the main loss function used by the machine learning module 154 could be the Binary Cross-Entropy (BCE) loss, which measures the average logarithmic difference between the predicted values (p(y_i)) and the actual value (y_i) in a binary classifier.

In some cases, the machine learning module 154 can optimize the hyperparameters for the best performing machine learning models so that such models could be further improved. The best performing models, based on the AUC ROC score and F1 score, can be evaluated against the pristine validation dataset. The best performing models can be re-trained using the approach described herein, and the best hyperparameters for each model can be obtained by using a grid search; where different combinations of hyperparameter values can be used to train each iteration to determine which combinations perform the best.

At block 216, the assessment module 156 generates a compendium of concise assessments comprising all (or a plurality) of possible combinations of the assessment features having a length equivalent to the output subset and having a classification accuracy within the predetermined accuracy level. The concise model classification accuracy can be assessed, for example, in two ways: (a) whether the concise model classification matches, or substantially matches within a predetermined tolerance, that obtained when all items of the sample assessment are used, and (b) whether the concise model classification matches the classification obtained by another assessment that is also designed to make the same classifications.

At block 218, the assessment module 156 outputs at least one of the concise assessments. In some cases, at least one of the concise assessments from the compendium of concise assessments is outputted to the database 146. In some cases, one of the concise assessments are outputted to the user interface 132 for each desired access of the assessment by the user. In some cases, each concise assessment outputted to the user interface 132 can be selected at random from the compendium of concise assessments; while in other cases, it can be selected using some set order. In some cases, upon completing the assessment, the assessment module 156 outputs the estimated output status (e.g., a specific class and the likelihood that the user belongs to such class) based on the completed assessment features in the concise assessment. Advantageously, this output status will correlate in accuracy with completing the longer sample assessment. In some cases, when the concise assessment classifies a user as belonging to a class of special interests at a certain likelihood level, the assessment module 156 can recommend that the user take a full sample assessment, or another assessment of the same kind, to verify the results.

In an example of an approach undertaken by the system 100, the system 100 can use machine learning techniques to generate an assessment on anxiety. Anxiety is one of the most common mental health issues affecting the world today. It is a normal human reaction to stressful situations, a fight-or-flight response inherited from human ancestors. Although having a moderated level of anxiety can be beneficial towards motivation, excessive amounts of anxiety and worry can be detrimental to one's day to day activities and productivity, and therefore be classified as a mental disorder, known as anxiety disorders. The most common form is Generalized Anxiety Disorder (GAD), which is the focus of this study. In the world today, as society creates increasing individual responsibility and demands, the sources of anxiety also broaden. Since anxiety disorders can arise from different triggers, they be classified into subtypes, which are discussed in detail below. Furthermore, anxiety disorders can also develop new mental health disorders, or worsen existing mental health concerns, including depression and schizophrenia.

Due to the negative effects of anxiety disorders on individual well-being and productivity, it is important to quickly recognize the symptoms of anxiety and monitor them regularly, while coming up with ways to mitigate their effects. Diagnosing and monitoring anxiety disorders is difficult for most individuals. Thus, there exists the need for a quick diagnostic and self-monitoring tool for anxiety disorders. Such a tool can be useful for both individuals and organizations for regular monitoring of mental wellness, and help lessen the burden on professional services.

Anxiety disorders are officially diagnosed using the definitions and criteria of the Diagnostic and Statistical Manual of Mental Disorders, Fifth Edition (DSM-5). A patient is generally considered to have an anxiety disorder if they have symptoms compatible with the DSM-5 criteria in the past 12 months. There are many other scales that are used to screen symptoms related to anxiety. All anxiety scales generally involve answering a questionnaire, where the responses for each question corresponds to a numerical value. Once complete, the values for all the questions are summed up to get a final score, which can be used to derive the likelihood and severity of anxiety symptoms. However, some of the existing tools for assessing anxiety disorders are inadequate for this purpose. They often require answering a lengthy survey and calculating a score to predict the presence and severity of anxiety disorders, which can often take up several minutes. This also makes unsuitable for regular monitoring since they are long. Also, patients can memorize the questions and therefore respond from memory rather than according to what they are experiencing at the moment. There is a substantial need for assessments to be shortened so they can be completed in about a minute or less and made non-repetitive for effective regular monitoring.

The system 100 can use machine learning techniques to address the substantial problem of assessment generation, as in the above for GAD. In an approach, machine learning can be used to identify items and/or questions in anxiety disorder assessment scales that are redundant, in order to generate a concise and accurate assessment. Machine learning techniques can also be used to find linear or non-linear combinations of items from assessment scales that can most accurately predict the presence of GAD. These subsets of items can be used for developing the shorter and more effective anxiety assessment that is efficient, convenient, and accurate.

In the example of generating a GAD rapid assessment, in some cases, an existing scale for assessing GAD is selected as a basis. For example, the Depression Anxiety Stress Scale (DASS) can be used. DASS-42 is a 42 item self-report scale designed to measure the negative emotional states of depression, anxiety, and stress. There are 14 items to measure each condition. For each item, a statement regarding the participant's feeling and/or experience is given, and the participant can select how closely they match the statement in the past week, with options of “never”, “sometimes”, “often”, and “almost always”, which correspond to scores 0, 1, 2, and 3, respectively. The total score for each condition is calculated by summing up the scores of all questions corresponding to each condition.

An input dataset of DASS survey results can be collected and received by the preprocessing module 150; where the data is labelled by the predicted outcome (normal or no anxiety, mild anxiety, moderate anxiety, and severe anxiety). The main dataset for this example is a compilation of a survey entries and results of the DASS-42 questionnaire collected from an internal online survey portal, which collected data worldwide. The dataset contains adults only (age 18), and there are approximately 40,000 participants in total. The dataset includes answers from all 42 items of the DASS-42 questionnaire, the participants' gender, age, and country of residence, as well as the anxiety, depression, and stress scores calculated from the DASS-42 scoring method. Since this example focused on anxiety, the depression and stress scores were discarded; however all 42 questions were examined by the system 100.

The input dataset can include raw data features; which can include demographics information (age, gender, ethnicity) in association with the survey answers to all the DASS questions. The preprocessing module 150 organizes and processes the raw data features. The columns that can be categorized in the input dataset are transformed into one-hot encoding representing each category. For instance, gender has two possible choices, male, and female, which can be transformed into two columns representing each gender, and only one column can be marked as 1 for every row (while the other columns are 0). For the country feature, to make it more generalized, it was reorganized into 3 ethnicities, “Eastern”, “Western”, and “Other”. “Eastern” consists of Asian countries, “Western” consists of Europe and the Americas, while “Other” encompasses the remainder of the countries. The transformed columns include the answers to all multiple-choice questions in the DASS-42 questionnaire (answers range from integers 0 to 3), gender (1 for male and 0 for female), and ethnicity (2 for Eastern, 1 for Western, and 0 for Other). All the scalar columns are then normalized using a z-score approach:

$$\\ Z = \frac{X - μ}{σ}$$

where x is the sample, μ is the population mean, and σ is the population standard deviation.

The preprocessing module 150 can then compute label columns for the input dataset. The DASS-42 questionnaire defines threshold scores for every severity level of anxiety (normal or no anxiety, mild, moderate, severe, and exceptional). A positive/negative anxiety status column is computed using the threshold score for the “moderate” category. If the DASS anxiety score exceeds the threshold, the status is positive (1), otherwise negative (0). Thus, a prediction can be made whether the participant has at least “moderate” anxiety levels defined by DASS. In the example input dataset, there are about 10,000 positive samples and about 30,000 negative samples. Based on the thresholds for each severity level, more columns can be computed in a one-hot encoding fashion: normal or no anxiety, mild, moderate, severe, and exceptional. In each row, only one of these columns can be 1 at a time, indicating the severity level of anxiety. All the other columns must be zero. The label columns can then be separated from the feature columns.

After checking the counts of rows with each label, the preprocessing module 150 balances the dataset by up sampling the positive test samples to match up with the number of negative samples. The up-sampling can randomly select rows from the specified categories and duplicates them in the dataset, so the row count matches the other categories. This can be used to ensure that the dataset is not biased towards a certain category.

The preprocessing module 150 can then partition the dataset into training, test, and pristine external validation datasets. In this example, a 10-fold cross validation can be used. 80% of the dataset is used for training the models, 10% of the dataset is used for internal model testing during training, and the remainder 10% is held out and used for model validation after completing the training. Throughout the training process, the training and internal test sets can be reshuffled and re-partitioned to ensure model consistency, while the validation set remains untouched. The reported performance measures of each model can be determined from the validation set.

The selection module 152 can then perform feature selection. Since the system 100 determines a reduce set of assessment questions required to accurately predict the presence of Generalized Anxiety Disorder, the selection module 152 uses feature selection techniques to check which questions from the DASS-42 questionnaire weigh more heavily in predicting the status (positive/negative) of anxiety. The Minimum Redundancy Maximum Relevance (MRMR) feature selection technique, using the Mutual Information Quotient (MIQ) criteria, was applied in this example to identify a top 10 most important questions that predict anxiety out of the 42 total questions in the DASS-42 questionnaire. MRMR selects the most relevant features based on pairwise correlations, or mutual information of each pair of variables in the dataset, while minimizing the redundancy between variables. In the present example, 10 questions are used because a preliminary analysis by the present inventors revealed that the top 10 most important questions was sufficient to make anxiety classification at the accuracy level of 90% Area Under the Curve (as described herein).

To serve as a reference, the selection module 152 used a second feature selection approach by fitting an Extra Tree Classifier for all the features and labels before ranking the most important features. The Extra Tree Classifier fits several randomized decision trees (i.e., extra-trees) on sub-samples of the dataset and averages the results. The feature importance was obtained by computing the normalized total reduction of the criterion brought by that feature, which is known as the Gini importance. The top 10 most important questions from DASS-42 are obtained by ranking the feature importance. These results are then combined with the top 10 questions from the MRMR selection to form a pool of 10 most important questions from DASS-42 to be used in the Generalized Anxiety Disorder assessment.

In this example, all 42 items, numbered from 1 to 42, from the DASS-42 questionnaire are included in the feature selection process to select the top 10 items. However, the DASS-42 items used to calculate the anxiety score, item numbers {2, 4, 7, 9, 15, 19, 20, 23, 25, 28, 30, 36, 40, 411}, are used as a baseline. additionally, common symptoms for Generalized Anxiety Disorder are used for reference. Selecting the top 10 most relevant items in DASS-42 using MRMR returned the item numbers {21, 7, 18, 11, 20, 4, 6, 1, 36, 40, 23}. 6 out of 10 items are used for calculating the DASS anxiety score, and the items are generally aligned with the common symptoms of Generalized Anxiety Disorder. Fitting the Extra Tree Classifier on all features and labels returned the item numbers [21, 36, 4, 13, 38, 17, 15, 26, 41, 28]from DASS-42.4 out of 10 items are used for computing the DASS anxiety score; 4 out of 10 items are also found to be among the 10 most important items from MRMR. Upon examination, most items generally match with the common symptoms of Generalized Anxiety Disorder. Upon combining the results, the 10 items numbered {21, 36, 4, 7, 13, 15, 17, 18, 11, 20, 6} were selected.

The machine learning module 154 then trains one or more machine learning models to predict the presence of Generalized Anxiety Disorder, based on the top 10 questions identified by the selection module 152, as well as demographics features in the dataset including age, gender, and ethnicity. The label is a binary column representing the presence of anxiety (e.g., 0 or 1), or in other cases the label can be multi-class (e.g., representing normal or no anxiety, and different levels of anxiety [mild, moderate, severe]. The training of the machine learning models can use the processed and class-balanced training dataset, consisting of 80% of the complete dataset.

Various suitable machine learning models can be used. For example, Regression (linear or logistic), Support Vector Machine (SVM), Random Forest (RF), and Naïve Bayes. Further, Artificial Neural Networks (ANN) can be used, for example, Multilayer Perceptron (MLP) and Convolutional Neutral Network (CNN). Further, gradient boosting or ensemble techniques, such as XGBoost, can be used. For the present GAD example, the present inventors tested the following models:
Logistic Regression (LR): A statistical model using a logistic function to model a categorical variable, commonly a binary dependant variable.
Support Vector Machine (SVM): A clustering technique that applies the statistics of support vectors to categorize unlabeled data, by deciding sets of hyperplanes that separate different classifications.
Random Forest (RF): An ensemble learning method for classification, regression and other tasks that operate by constructing a multitude of decision trees at training time and outputting the class that is the mode of the classes (classification) or mean prediction (regression) of the individual trees.
Multilayer Perceptron (MLP): A type of feedforward artificial neural network (ANN) that is composed of multiple layers of nodes with biases and activation thresholds and edges with weights.
Extreme Gradient Boosting (XGBoost): A variation of the gradient boosting technique, designed to increase system performance; combines Stochastic Gradient Boosting with Regularized Gradient Boosting.

The primary loss function used to evaluate the above models during training was the Binary Cross-Entropy (BCE) loss; which measures the average logarithmic difference between the predicted values and the actual value in a binary classification:

[symbol]

To evaluate the performance of each machine learning model, the model is run on a pristine validation dataset (10% of the complete dataset) that is balanced between different classes, and a series of metrics are taken:
Area Under Curve (AUC) score of Receiver Operating Characteristic (ROC) curve: Measures the diagnostic ability of a binary classifier system by plotting the true positive (TP) rate against the false positive (FP) rate at various threshold settings. The ROC is a probability curve, and the AUC represents the degree or measure of separability.
Precision: Number of true positives (TP) over the number of true positives (TP) plus the number of false positives (FP):

$$\\ Precision = \frac{T P}{T P + F P}$$

Recall: Also known as Sensitivity, defined as the number of true positives (TP) over the number of true positives (TP) plus the number of false negatives (FN):

$$\\ Recall = \frac{T P}{T P + F N}$$

F1 Score: Harmonic mean of precision and recall, used to measure the performance of a model's classification ability over both positive and negative cases:

$$\\ F 1 = 2 \times \frac{Precision \times Recall}{Precision + Recall} = \frac{TP}{TP + \frac{1}{2} ( FP + FN )}$$

In this example experiment, the machine learning module 154 was used to evaluate the models by determining how many questions from the DASS-42 it would take for the respective model to predict the presence of anxiety at a reasonable accuracy. To do so, models were trained for different permutations of questions selected from the top 10 questions selected from the MRMR approach described above, along with the demographics (age, gender, ethnicity), starting from permutations of 1 question to 8 questions from DASS. For each number of questions, 10 different permutations of questions were run, which were kept the same across all models, for every number of questions. For every permutation, 50 models (ensembles) were trained on 50 different partitions (reshuffling) of the training dataset and evaluated. All models were trained on their default hyperparameters and stopping criteria, shown in TABLE 1, from the imported libraries to ensure consistency. The average AUC scores of ROC curve and F1 scores were evaluated on the validation dataset for each model across all ensembles, as well as the 95% confidence interval for each across 10 permutations.

TABLE 1

| | | Hyperparameters and | |
|---|---|---|---|
| Model | Python Library and Class | Default Values | |
| Logistic | Scikit-learn | penalty | I2 |
| Regression | sklearn.linear_model.Lo- | dual | False |
| | gisticRegression | tol (Tolerance for | 1e−4 |
| | | stopping criteria) | |
| | | C (Inverse of regu- | 1 |
| | | larization strength) | |
| | | fit_intercept | True |
| | | intercept_scaling | 1 |
| | | class_weight | None |
| | | random_state | None |
| | | solver | lbfgs |
| | | max_iter | 100 |
| | | multi_class | auto |
| | | verbose | 0 |
| | | warm_start | False |
| | | n_jobs | None |
| | | I1_ratio | None |
| Support | Scikit-learn | C (Inverse of regu- | 1 |
| Vector | sklearn.svm.SVC | larization strength) | |
| Machine | | kernel | rbf |
| | | degree | 3 |
| | | gamma | scale |
| | | coef0 | 0 |
| | | shrinking | True |
| | | probability | False |
| | | tol (Tolerance for | 1e−3 |
| | | stopping criteria) | |
| | | cache_size | 200 |
| | | class_weight | None |
| | | verbose | False |
| | | max_iter | −1 |
| | | decision_func- | ovr |
| | | tion_shape | |
| | | break_ties | False |
| | | random_state | None |
| Random | Scikit-learn | n_estimators | 100 |
| Forest | sklearn.ensemble.Ran- | criterion | gini |
| | domForestClassifier | max_depth (max- | None |
| | | imum depth of the | |
| | | tree) | |
| | | min_samples_split | 2 |
| | | min_samples_leaf | 1 |
| | | min_weight_frac- | 0 |
| | | tion_leaf | |
| | | max_features | auto |
| | | max_leaf_nodes | None |
| | | min_impurity_de- | 0 |
| | | crease | |
| | | min_impurity_split | None |
| | | bootstrap | True |
| | | oob_score | False |
| | | n_jobs | None |
| | | random_state | None |
| | | verbose | 0 |
| | | warm_start | False |
| | | class_weight | None |
| | | ccp_alpha | 0 |
| | | max_samples | None |
| Multilayer | PyTorch | epochs | 10 |
| Perceptron | torch.nn.Module * | batch size | 100 |
| | | learning rate | 0.01 |
| | | number of hidden | 1 |
| | | layers | |
| | | hidden layer size | 100 |
| | | bias | 1 |
| | | optimizer | SGD |
| | | criterion (loss | BCE |
| | | function) | |
| | | activation function | tanh |
| XGBoost | XGBoost | n_estimators | 100 |
| | xgboost.xgbClassifier | use_label_encoder | False |
| | | max_depth | 6 |
| | | learning_rate | 0.1 |
| | | verbosity | 0 |
| | | objective | linear |
| | | booster | gbtree |
| | | tree_method | auto |
| | | n_jobs | None |
| | | gamma | 0 |
| | | min_child_weight | 1 |
| | | max_delta_step | 0 |
| | | subsample | 1 |
| | | colsample_bytree | 1 |
| | | colsample_bylevel | 1 |
| | | colsample_bynode | 1 |
| | | reg_alpha | 0 |
| | | reg_lambda | 1 |
| | | scale_pos_weight | 1 |
| | | base_score | 0.5 |
| | | random_state | 0 |

The machine learning module 154 optimized the hyperparameters for the best performing models. Each model is trained using the same procedure as above. The best hyperparameters for each model is obtained by using a grid search, where different combinations of hyperparameter values are used to train each iteration to determine which combinations are the best. In addition to the AUC and F1 scores, precision and recall are also reported for this comparison. The best models at the end were chosen for implementation into the anxiety rapid assessment and validated.

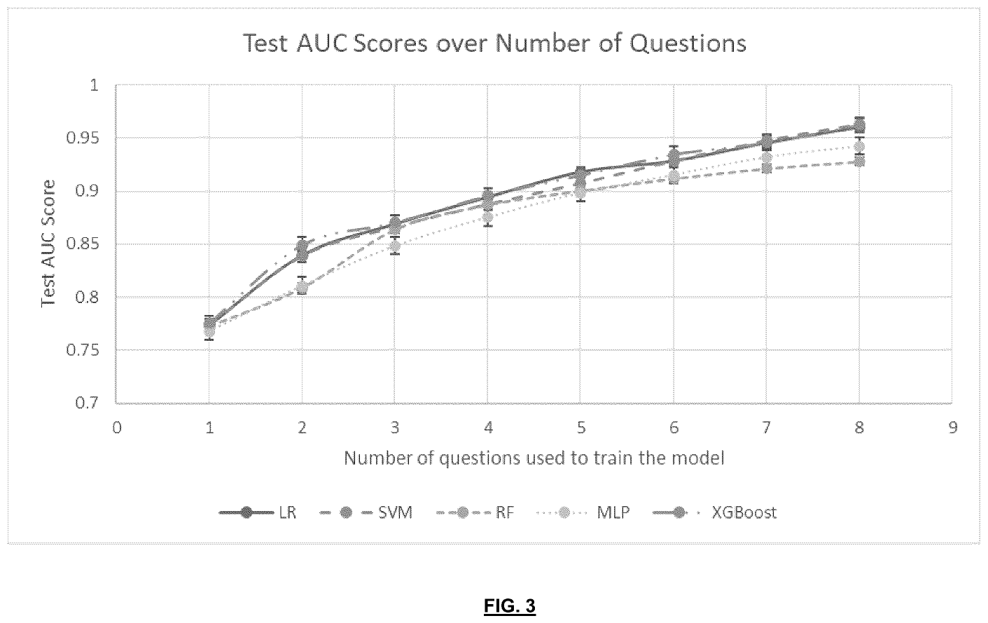

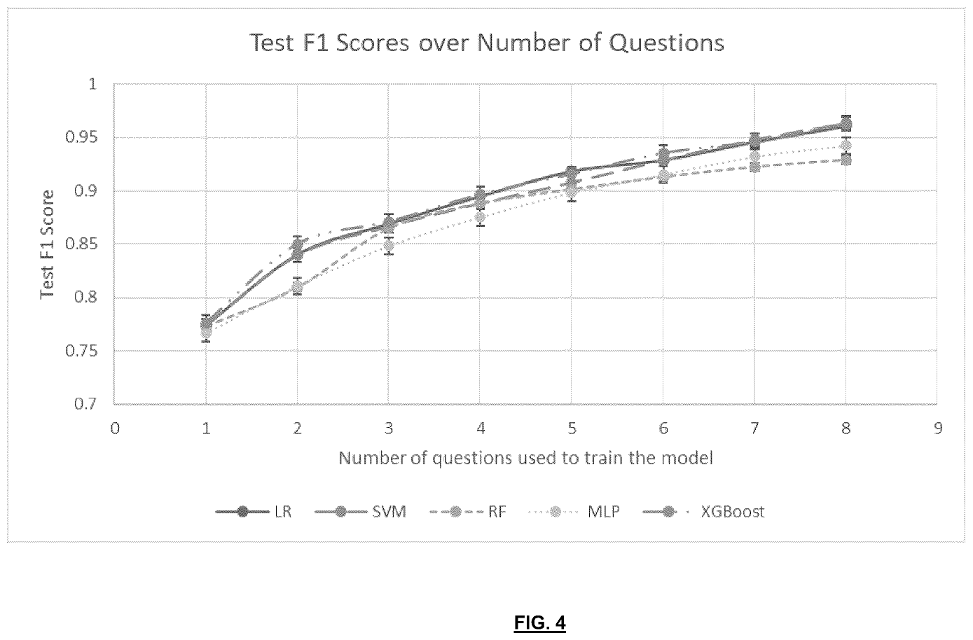

For this example, after training all the machine learning models, the results can be plotted. FIG. 3 shows the AUC of ROC scores, and FIG. 4 shows the F1 scores, both for permutations of 1 question to 8 questions, averaged over 50 ensembles for each model and permutation, over 10 permutations for each number of questions, and across the five machine learning techniques outlined above, on the pristine validation dataset. The error bars represent the range of the 95% confidence interval of each metric over the 10 permutations.

Looking at the graphs of FIG. 3 and FIG. 4, models performed better as the number of questions in the training features are increased, but the amount of performance improvement diminishes with more questions. On average, the test AUC scores and F1 scores for all models exceed 90% at 5 questions, which is the desired accuracy threshold defined above. Thus, a minimum of 5 questions/items from DASS-42 can be used for rapid assessment for the presence of anxiety. The best performing models on average appear to be Logistic Regression and XGBoost. To explore more trends, a more detailed table of results for all the models run on permutations of 5 questions is presented in TABLE 2.

TABLE 2

| | AUC | Standard | 95% Confidence | F1 | Standard | 95% Confidence |
|---|---|---|---|---|---|---|
| Model | Score | Deviation | Interval | Score | Deviation | Interval |
| Logistic | 91.80% | 0.43% | 91.53%-92.06% | 91.83% | 0.43% | 91.63%-92.16% |
| Regression | | | | | | |
| Support | 90.70% | 0.96% | 90.11%-91.30% | 90.76% | 0.95% | 90.17%-91.35% |
| Vector | | | | | | |
| Machine | | | | | | |
| Random | 90.03% | 0.64% | 89.63%-90.43% | 90.10% | 0.64% | 89.70%-90.50% |
| Forest | | | | | | |
| Multilayer | 89.80% | 1.35% | 88.96%-90.64% | 89.79% | 1.36% | 88.95%-90.63% |
| Perceptron | | | | | | |
| XGBoost | 91.50% | 1.14% | 90.79%-92.30% | 91.55% | 1.14% | 90.84%-92.35% |

Based on TABLE 2, Logistic Regression is the best performing model based on the metrics of AUC score and F1 score, while XGBoost is a close second. However, their 95% confidence intervals overlap, and XGBoost has a slightly higher upper bound for both AUC and F1-score, despite the larger variance between different permutations of questions. This indicates the potential for XGBoost to exceed the performance of Logistic Regression. Since all models are trained on their default settings and hyperparameters, the performance of XGBoost and Logistic Regression techniques can be further defined by tuning their hyperparameters. The next step of the machine learning is to optimize the hyperparameters for XGBoost and Logistic Regression. The best model test results from each of the above two techniques are summarized in TABLE 3. In addition to the metrics presented in TABLE 2, precision and recall are also included for this comparison.

| | AUC | Standard | | F1 | Standard | | | |
|---|---|---|---|---|---|---|---|---|
| Model | Score | Deviation | 95% CI | Score | Deviation | 95% CI | Precision | Recall |
| Logistic | 91.90% | 0.46% | 91.63% | 91.93% | 0.47% | 91.73% | 91.98% | 91.91% |
| Regression | | | 92.17% | | | 92.26% | | |
| XGBoost | 92.37% | 0.81% | 91.87% | 92.44% | 0.80% | 91.95% | 92.52% | 92.45% |
| | | | 92.87% | | | 92.94% | | |

XGBoost performs better than Logistic Regression on all metrics after optimizing for hyperparameters. There is still more variance in the results of XGBoost, but the 95% confidence interval for AUC and F1 scores mostly exceed the ranges for Logistic Regression. The precision and recall metrics are also slightly in favour of XGBoost. Thus, for this example, focus was palced on improving the performance of the XGBoost technique on permutations of 5 questions from the top 10 most relevant questions in DASS-42 and demographics features (age, gender, and ethnicity), and implementing the model into the anxiety rapid assessment.

Further improvements were made to the XGBoost model by tuning the max depth and the number of estimators. The number of ensembles for each model was also reduced from 50 to 10 to improve memory performance, while sacrificing little in terms of model performance. This resulted in an average validation AUC score of 92.26% and F1 score of 92.33% across 10 permutations of 5 questions. Finally, out of the 10 permutations, the top 5 permutations that produced the highest AUC and F1 scores were chosen for implementation into the anxiety rapid assessment. These 5 permutations have an average validation AUC ROC score of 93.01% and F1 score of 92.92%, as, well as a Precision of 93.04% and Recall of 92.97%. The best permutation has a validation AUC score of 93.74% and F1 score of 93.63%.

In this example, validation by the machine learning module 154 consists of two steps. The first step is entering a fixed set of responses, with permutations of different values for different demographics and DASS items, for all sets of the questionnaire and checking if the predicted probability of Generalized Anxiety Disorder is realistic. It would simulate different combinations of responses that a user would input into the assessment. The second step is to enter several different responses from the pristine validation dataset, and tally the average binary prediction accuracy (AUC ROC score and F1 score) to match that of the pristine dataset when tested on the raw models, where the numbers must match exactly.

The outcome of this example includes machine learning prediction models that accurately predict the likelihood of a particular anxiety class given demographics and answers from a relatively short survey. The anxiety class can include, for example, normal (or no anxiety) and anxiety [mild, moderate, severe]. Each survey can be completed in about a minute or less. In this example, the assessment module 156 can output the GAD assessment as a web application, which has a user interface and uses the selected machine learning model. The assessment module 156 selects a different set of questions and/or items each time to prevent memorization. The deployment of this assessment enables individuals the ability to self-monitor their anxiety level, quickly and frequently.

In this example, the present inventors built the anxiety rapid assessment in the form of a web application. The web application allowed a user to answer a short survey including age, gender, ethnicity, and a set of five questions/items from the DASS-42 questionnaire, a total of 8 items. It takes approximately a minute or less for the user to complete one survey. Once submitted, the system determines the likelihood that the user has a particular generalized anxiety level using the assessment module 156. The assessment module 156 randomizes the five DASS questions every time based on which questions the models require, with a total of 5 question sets. The models implemented use the optimized XGBoost technique, and ensembles of 10 different models for every set of questions. There are 5 different sets of questions in total. The present embodiments encompass multiple advantages compared to existing approaches for assessing generalized anxiety levels. First, it was found that very few numbers of questions are required to predict the existence of generalized anxiety at a sufficient accuracy. Having too many questions on an assessment is redundant, and often makes it too long and boring for the patients. Second, the assessment utilizes multiple sets of different questions to assess Generalized Anxiety Disorder instead of repeating the same sets of questions over and over. This makes it less likely for the patients to get bored of answering the same questions repeatedly, as well as the risk of memorizing the answers over many attempts. Third, an extension of the current approach can be carried out by training a multi-class machine learning classifier, which would allow the machine learning models to predict more labels such as severity, from normal to mild to severe. In this case, the system 100 can use a multi-class output status instead of a binary status, and train computational models to produce multi-class predictions and likelihood ratios (e.g., normal or no anxiety, mild anxiety, moderate anxiety, and severe anxiety). Finally, this study shows that predictors not traditionally suited to predict anxiety can be used to predict the presence of generalized anxiety. In the DASS scale used for this study, it was found that more than half of the top 10 most important items is not specific for calculating the DASS anxiety score. It shows that the items within the DASS assessment can carry more information than just one condition, and they can be used to assess other psychological conditions in addition to anxiety, depression, or stress.

The present embodiments can improve monitoring of generalized anxiety by reducing its complexity and making it much more convenient. The generalized anxiety rapid assessment can be useful for monitoring anxiety at a personal level and organizational level. At the individual level, it can be help individuals identify their symptoms for Generalized Anxiety Disorder and determine when to seek professional help. At the organizational level, this tool can enable monitoring of the members' mental wellness, and help identify systematic issues contributing to anxiety disorders. It can be useful in high stress environments such as schools and businesses. This technique can be applied to develop rapid assessment tools to predict depression and stress in DASS-42, as well as other anxiety disorders and psychological disorders, which would help with identifying and distinguishing between these disorders.

Currently, there are other ways to assess Generalized Anxiety Disorder and other anxiety disorders aside from using a defined scale with a questionnaire. Wearable devices have been used to monitor symptoms of anxiety in real time by monitoring changes in physiological parameters; such as with an electrocardiogram (ECG) signal. These changes can imply variations in the cardiovascular and nervous systems that a person experiences when they go through an anxiety episode. The techniques of the present embodiments can be used in conjunction with data collected from wearable devices to produce more accurate results for predicting anxiety levels and anxiety disorders.

While the present disclosure uses the example of assessments to determine generalized anxiety levels, it is understood that the present embodiments can be used to construct any efficient, convenient, and accurate short assessment based on a longer base assessment. Particularly, by selecting the most important features or items that carry the most predictive information about a queried condition. An illustrative and non-limiting set of such applications can include:
Personality assessments (e.g., the Big Five Personality Traits).
Ability tests, such as, IQ tests and aptitude tests (e.g., music ability)
Achievement tests, such as Graduate Record Examinations, Medical College Admission Test, Scholastic Assessment Test, and other examinations.
Survey of attitude and opinions (e.g., voting intention, marketing, health and lifestyle).

While the present disclosure uses the example of assessments to determine generalized anxiety levels based on a subset of items of a scale to predict classifications when all items of the scale are used, it is understood that this approach can be used to predict scores or classification of generalized anxiety levels or generalized anxiety disorder as assessed by other generalized anxiety scales or diagnosis by trained and qualified clinicians.

Although the invention has been described with reference to certain specific embodiments, various modifications thereof will be apparent to those skilled in the art without departing from the spirit and scope of the invention as outlined in the claims appended hereto. The entire disclosures of all references recited above are incorporated herein by reference.

1. A method for concise assessment generation using machine learning, the method executed on one or more processors, the method comprising:
receiving an input dataset comprising completed sample assessments, the sample assessments each comprise assessment features for determining an output status and an associated likelihood of the output status;
selecting a subset of the assessment features by determining which assessment features of the sample assessment weigh more heavily in predicting the output status;
predicting, using a machine learning model, a quantity of assessment features required to achieve at least a predetermined classification accuracy of an output of the assessment, the machine learning model trained using the selected subset of assessment features and the weighting of such assessment features; and
outputting one or more concise assessments, each concise assessment comprising the quantity of assessment features and having a classification accuracy of at least the predetermined classification accuracy.

2. The method of claim 1, further comprising encoding and normalizing the assessment features.

3. The method of claim 2, further comprising up-sampling positive sample assessments to match the number of negative sample assessments.

4. The method of claim 1, wherein selecting a subset of the assessment features comprises performing Minimum Redundancy Maximum Relevance (MRMR) feature selection using Mutual Information Quotient (MIQ) criteria.

5. The method of claim 4, wherein selecting a subset of the assessment features further comprises performing feature selection by fitting an Extra Tree Classifier for the assessment features and ranking importance of the assessment features.

6. The method of claim 5, wherein the MRMR feature selection is combined with the Extra Tree Classifier feature selection.

7. The method of claim 1, wherein the machine learning model is trained using multiple different permutations of assessment features from the selected subset.

8. The method of claim 7, wherein training of the machine learning model begins with permutations of one of the assessment features followed by permutations of increasing numbers of assessment features until the predetermined accuracy is achieved.

9. The method of claim 1, wherein the one or more concise assessments comprise a compendium of all the concise assessments that comprise the quantity of assessment features and have a classification accuracy of at least the predetermined classification accuracy.

10. The method of claim 1, wherein classification accuracy of the concise assessments is determined based on whether the classifications outputted by the concise assessments match, or substantially match within a predetermined tolerance, that obtained when all items of the sample assessment are used.

11. A system for concise assessment generation using machine learning, the system comprising one or more processors and a data storage, the data storage comprising instructions for the one or more processors to execute:
a preprocessing module to receive an input dataset comprising completed sample assessments, the sample assessments each comprise assessment features for determining an output status and an associated likelihood of the output status;
a selection module to select a subset of the assessment features by determining which assessment features of the sample assessment weigh more heavily in predicting the output status;
a machine learning module to predict, using a machine learning model, a quantity of assessment features required to achieve at least a predetermined classification accuracy of an output of the assessment, the machine learning model trained using the selected subset of assessment features and the weighting of such assessment features; and
an assessment module to output one or more concise assessments, each concise assessment comprising the quantity of assessment features and having a classification accuracy of at least the predetermined classification accuracy.

12. The system of claim 11, wherein the selection module further encodes and normalizes the assessment features.

13. The system of claim 12, wherein the selection module further up-samples positive sample assessments to match the number of negative sample assessments.

14. The system of claim 11, wherein selecting a subset of the assessment features comprises performing Minimum Redundancy Maximum Relevance (MRMR) feature selection using Mutual Information Quotient (MIQ) criteria.

15. The system of claim 14, wherein selecting a subset of the assessment features further comprises performing feature selection by fitting an Extra Tree Classifier for the assessment features and ranking importance of the assessment features.

16. The system of claim 15, wherein the MRMR feature selection is combined with the Extra Tree Classifier feature selection.

17. The system of claim 11, wherein the machine learning model is trained using multiple different permutations of assessment features from the selected subset.

18. The system of claim 17, wherein training of the machine learning model begins with permutations of one of the assessment features followed by permutations of increasing numbers of assessment features until the predetermined classification accuracy is achieved.

19. The system of claim 11, wherein the one or more concise assessments comprise a compendium of all the concise assessments that comprise the quantity of assessment features and have a classification accuracy of at least the predetermined classification accuracy

20. The system of claim 11, wherein classification accuracy of the concise assessments is determined based on whether the classifications outputted by the concise assessments match, or substantially match within a predetermined tolerance, that obtained when all items of the sample assessment are used.
